(19) United States
(12) Patent Application Publication
(10) Pub. No.: US 2026/0146923 A1
(43) Pub. Date: May 28, 2026

(54) APPARATUS AND METHOD FOR FLUID SAMPLING

(71) Applicant: atdepth MRV, Inc., Dover, DE (US)

(72) Inventors: Souha El Mousadik, Cambridge, MA (US); Carlos Muñoz-Royo, Cambridge, MA (US); Raphaël Ouillon, Geneva (CH)

(73) Assignee: atdepth MRV, Inc., Dover, DE (US)

(21) Appl. No.: 19/396,868

(22) Filed: Nov. 21, 2025

Related U.S. Application Data
(60) Provisional application No. 63/724,508, filed on Nov. 25, 2024.

(51) Int. Cl. G01N 1/14; G01N 1/34; G01N 1/38; G01N 33/18

(52) U.S. Cl. CPC G01N 1/14; G01N 1/34; G01N 1/38; G01N 33/18

(57) ABSTRACT

An apparatus and method for fluid sampling is provided. The apparatus includes: a sampling chamber, configured to receive a fluid sample; at least one controllable inlet fluidically coupled to the sampling chamber; a pump, configured to draw fluid through the inlet and into the sampling chamber; at least one sensor, disposed to measure at least one property of the fluid sample within the sampling chamber; and a processor, configured to control operation of the inlet, the pump, and the at least one sensor to obtain a measurement of the fluid sample following equilibration of the fluid sample.

### RELATED APPLICATIONS

This application claims the benefit of the following U.S. Provisional applications, each of which is herein incorporated by reference in its entirety.

| SERIAL | FILING | |
|---|---|---|
| NUMBER | DATE | TITLE |
| 63724508 | Nov. 25, | FLUID SAMPLING CHAMBER |
| (ADV.0401P) | 2024 | |

This application is related to the following co-pending U.S. patent applications, each of which has a common assignee and common inventors, the entireties of which are herein incorporated by reference.

| SERIAL | FILING | |
|---|---|---|
| NUMBER | DATE | TITLE |
| (̅A̅D̅V̅.̅0̅4̅0̅2̅)̅ | | FLUID SAMPLING CHAMBER |

### BACKGROUND OF THE INVENTION

### Field of the Invention

This invention relates in general to the field of fluid property measurements, and more particularly to a fluid sampling chamber.

### Description of the Related Art

For most of history, primary utilization of the ocean has revolved around fishing and transportation. In more recent years new uses are emerging such as carbon removal, renewable energies, aquaculture (e.g., seaweed farming), innovative transportation solutions, coastal resilience, biodiversity credits, and others.

As one skilled in the art will appreciate, these so-called “blue industries” impact or directly rely on physical and biogeochemical processes in the ocean where human interventions initially have very local effects, yet those effects can then extend very far. Because ocean monitoring is costly and sparse quantification of those effects through modeling and monitoring is critical to optimize operations and better predict impacts. With particular regard to carbon dioxide (CO2) removal, existing modeling and monitoring techniques offer low certainty quantification of CO2, thus resulting in less trust and slower adoption by potential stakeholders. As one skilled in the art will also appreciate, an ocean carbon dioxide removal (CDR) system is deployed to remove CO2 from a local body of water, but complex ocean processes (e.g., turbulent mixing, ocean currents, buoyancy, etc.) transport, dilute, and diffuse the CO2-depleted water. When this CO2-depleted water comes in contact with the surface, atmospheric CO2 is absorbed, leading to effective carbon removal. Ocean CDR offers great potential for battling climate change because it is almost infinitely scalable.

With the advent and availability of powerful graphics processing units (GPU's), the present inventors have developed robust equation-based models and measurement techniques that enable high certainty quantification of human interventions. These models run on GPU's continuously and in real-time, where global, regional, and local high-resolution simulations and nested and all relevant physical and biogeochemical processes are resolved to produce transparent, continuous and real-time data and analytics that are more accurate and reliable than that which has been heretofore provided.

Carbon removal techniques have matured over more recent years to include requirements for measurement, reporting, and verification (MRV), namely a set of systematic processes that are employed to track, document, and validate greenhouse gas emissions and reductions to ensure the credibility and accuracy of carbon removal projects. These processes are required to quantify with certainty whether or not implemented human interventions result in promised CDR. Because ocean conditions (e.g., global currents, near-field turbulent mixing, buoyancy-driven flows) are continually evolving, these MRV processes must run continuously over the life of the operation. By improving MRV accuracy and reliability, delay-free issuance of carbon credits is enabled. This disclosure focuses on the measurement aspect and challenges of ocean MRV.

As one skilled in the art will further appreciate, in highly variable environments, it is not possible to use forced diffusion-based instruments and many other sensors to make accurate measurements of many fluid parameters including, but not limited to, dissolved oxygen, total alkalinity, chlorophyll, temperature, conductivity, turbidity, partial pressure carbon dioxide (pCO2), and pH in a fluid where the equilibration time of at least one of the sensors used to measure those properties is significantly greater than the temporal variability of a corresponding parameter that is measured by the at least one of the sensors. In the case where the fluid is sea water, there is no perfect location to sample properties of interest due to significant differences in temporal variability and property heterogeneity from one location to the next. Such is true of the atmosphere, rivers, lakes, oceans, and the like where environmental factors (e.g., gravity, temperature, wind, etc.) cause constant change.

The present inventors have noted that current techniques for measuring properties in such fluids include using Niskin bottles, avoiding locations that are known to exhibit high heterogeneity and temporal variability of fluid properties, and merely assuming that a given sensor will provide an average measurement of the parameter of interest. These techniques are disadvantageous and limiting for many reasons, a few of which are discussed below.

As one skilled in the art will concur, for most applications investigators are not at liberty to select a perfect location to take fluid samples. For instance, it may be required to measure properties of a fluid resulting from a process performed on that fluid by a system in fixed location such as a power plant, where it is required to measure the properties in the cooling water outfalls of the plant—areas that notoriously exhibit high temporal variability and heterogeneity.

Likewise, using Niskin bottles to obtain samples is a technique that is labor-intensive, costly, and requires skilled and active personnel. In addition, this technique entails risks to the human life when employed to obtain the samples in the open ocean or in coastal environments. Furthermore, this technique requires a rigorous laboratory set up and careful sample manipulation to avoid contamination of the collected samples, which require complex preservation and storage methods to avoid biasing measurements. For example, some of the water quality parameters are temperature dependent, thus necessitating facilities having temperature-controlled environments to replicate exact ocean conditions, which additionally requires that water temperature be measured during sample collection. Additionally, most water samplers are very large and cannot sample targeted water volumes accurately.

Similarly, mere reliance that a given sensor will provide an average measurement of the parameter of interest is highly uncertain because parameter values are continually changing. Consequently, most sensors in these environments cannot properly equilibrate, thus leading to completely incorrect measurements.

Accordingly, what is needed is a system and method for measuring one or more fluid properties in scenarios where temporal variability of those properties in a solution of interest is not commensurate with the equilibration time of sensors employed to measure those properties.

Also, what is needed is a fluid sampling mechanism for use close to turbulent effluent discharge plumes that is more accurate than that which has heretofore been provided.

In addition, what is needed is a configurable fluid sampling system and method that enables independent measurements from differing depths.

What is further needed is a fluid sampling mechanism that is maintainable and resilient to open ocean conditions such as clogging and biofouling.

### SUMMARY OF THE INVENTION

The present invention, among other applications, is directed to solving the above-noted problems and addresses other problems, disadvantages, and limitations of the prior art. In one embodiment, an apparatus for fluid sampling is provided. The apparatus includes: a sampling chamber, configured to receive a fluid sample; at least one controllable inlet fluidically coupled to the sampling chamber; a pump, configured to draw fluid through the inlet and into the sampling chamber; at least one sensor, disposed to measure at least one property of the fluid sample within the sampling chamber; and a processor, configured to control operation of the inlet, the pump, and the at least one sensor to obtain a measurement of the fluid sample following equilibration of the fluid sample.

One aspect of the present invention contemplates a fluid sampling chamber for in-situ measurement of heterogeneous aquatic fluids, including: a sealed measuring chamber comprising a flexible bladder, configured to define an internal sampling volume; at least one controllable sampling inlet coupled to the flexible bladder; a pump, configured to flush the flexible bladder and draw a fluid sample into the internal sampling volume; a mixer, positioned to homogenize the fluid sample within the internal sampling volume; at least one sensor having an equilibration time greater than temporal variability of at least one fluid property external to the chamber; a cleaner; at least one unidirectional flow valve configured to seal the internal sampling volume during equilibration; and a processor configured to: perform a flushing cycle prior to sampling; fill the internal sampling volume with the fluid sample; activate the mixer to homogenize the fluid sample; allow the at least one sensor to equilibrate while the internal sampling volume is sealed; obtain at least one measurement from the at least one sensor; and activate the cleaner between sampling operations.

Another aspect comprehends a method for sampling a fluid, including: drawing a fluid sample into a sampling chamber through at least one controllable inlet; allowing at least one sensor disposed in the sampling chamber to measure at least one property of the fluid sample; and controlling operation of the inlet, a pump, and the at least one sensor using a processor to obtain the measurement.

### BRIEF DESCRIPTION OF THE DRAWINGS

These and other objects, features, and advantages of the present invention will become better understood with regard to the following description, and accompanying drawings where:

FIG. 1 is an environmental diagram illustrating an exemplary carbon dioxide removal system according to the present invention;

FIG. 2 is a diagram depicting a top view, a side view, and a perspective view of an exemplary fluid sampling chamber according to the present invention;

FIG. 3 is a block diagram of an exemplary fluid sampling chamber according to the present invention; and

FIG. 4 is a flow diagram showing an exemplary method for fluid sampling according to the present invention.

### DETAILED DESCRIPTION

Exemplary and illustrative embodiments of the invention are described below. It should be understood at the outset that although exemplary embodiments are illustrated in the figures and described below, the principles of the present disclosure may be implemented using any number of techniques, whether currently known or not. In the interest of clarity, not all features of an actual implementation are described in this specification, for those skilled in the art will appreciate that in the development of any such actual embodiment, numerous implementation specific decisions are made to achieve specific goals, such as compliance with system-related and business-related constraints, which vary from one implementation to another. Furthermore, it will be appreciated that such a development effort might be complex and time-consuming, but would nevertheless be a routine undertaking for those of ordinary skill in the art having the benefit of this disclosure. Various modifications to the preferred embodiment will be apparent to those skilled in the art, and the general principles defined herein may be applied to other embodiments. Therefore, the present invention is not intended to be limited to the particular embodiments shown and described herein, but is to be accorded the widest scope consistent with the principles and novel features herein disclosed.

The present invention will now be described with reference to the attached figures. Various structures, systems, and devices are schematically depicted in the drawings for purposes of explanation only and so as to not obscure the present invention with details that are well known to those skilled in the art. Nevertheless, the attached drawings are included to describe and explain illustrative examples of the present invention. Unless otherwise specifically noted, articles depicted in the drawings are not necessarily drawn to scale.

The words and phrases used herein should be understood and interpreted to have a meaning consistent with the understanding of those words and phrases by those skilled in the relevant art. No special definition of a term or phrase (i.e., a definition that is different from the ordinary and customary meaning as understood by those skilled in the art) is intended to be implied by consistent usage of the term or phrase herein. To the extent that a term or phrase is intended to have a special meaning (i.e., a meaning other than that understood by skilled artisans) such a special definition will be expressly set forth in the specification in a definitional manner that directly and unequivocally provides the special definition for the term or phrase. As used in this disclosure, “each” refers to each member of a set, each member of a subset, each member of a group, each member of a portion, each member of a part, etc.

Applicants note that unless the words “means for” or “step for” are explicitly used in a particular claim, it is not intended that any of the appended claims or claim elements are recited in such a manner as to invoke 35 U.S.C. § 112(f).

### Definitions

Integrated Circuit (IC): A set of electronic circuits fabricated on a small piece of semiconductor material, typically silicon. An IC is also referred to as a chip, a microchip, or a die.

Central Processing Unit (CPU): The electronic circuits (i.e., “hardware”) that execute the instructions of a computer program (also known as a “computer application,” “application,” “application program,” “app,” “computer program,” or “program”) by performing operations on data, where the operations may include arithmetic operations, logical operations, or input/output operations. A CPU may also be referred to as a “processor.”

Module: As used herein, the term “module” may refer to, be part of, or include an application specific integrated circuit (ASIC), an electronic circuit, a processor (shared, dedicated, or group) and/or memory (shared, dedicated, or group) that execute one or more computer programs, a combinational logic circuit, and/or other suitable components that provide the described functionality.

Microprocessor: An electronic device that functions as a CPU on a single integrated circuit. A microprocessor receives digital data as input, processes the data according to instructions fetched from a memory (either on-die or off-die), and generates results of operations prescribed by the instructions as output. A general-purpose microprocessor may be employed in a desktop, mobile, or tablet computer, and is employed for uses such as computation, text editing, multimedia display, and Internet browsing. A microprocessor may also be disposed in an embedded system to control a wide variety of devices including appliances, mobile telephones, smart phones, and industrial control devices.

Internet: The Internet is a global wide area network connecting computers throughout the world via a plurality of high-bandwidth data links which are collectively known as the Internet backbone. The Internet backbone may be coupled to Internet hubs that route data to other locations, such as web servers and Internet Service Providers (ISPs). The ISPs route data between individual computers and the Internet and may employ a variety of links to couple to the individual computers including, but not limited to, cable, DSL, fiber, and Wi-Fi to enable the individual computers to transmit and receive data over in the form of email, web page services, social media, etc. The Internet may also be referred to as the world-wide web or merely the web.

Equilibration: The process of allowing a sample (typically a sample of seawater and a small volume of air) to reach a state of quiescence or equilibrium so that accurate measurements of dissolved gases (like carbon dioxide or oxygen) can be made with assurance that a concentration (specifically, the partial pressure) of a gas in the air phase is the same as the concentration of that gas dissolved in the water phase at the same temperature and pressure. Many present-day sensors require approximately 10-15 minutes to equilibrate.

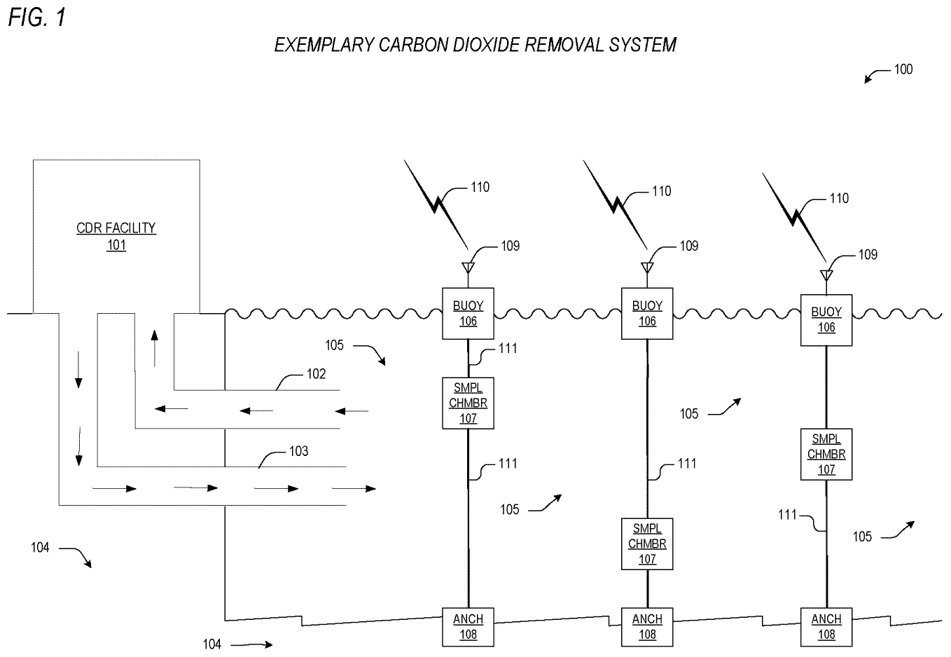

In view of the above background discussion on ocean CDR and associated techniques employed to measure and monitor fluid samples associated with human interventions, a discussion of the present invention will now be presented with reference to FIGS. 1-4. The present invention overcomes the challenges and limitations of those techniques by providing an system and method for capturing and measuring properties of heterogenous fluid samples that is more accurate and reliable than that which has heretofore been provided.

Turning to FIG. 1, an environmental diagram is presented illustrating an exemplary carbon dioxide removal system 100 according to the present invention. The CDR system 100 may comprise a CDR facility 101 deployed on land 104 that ingests local ocean water 105 via an intake port 102. The CDR facility 101 may remove CO2 from the ingested water 105 by any number of conventional means that include, but are not limited to, forced or induced draft decarbonization, vacuum de-aeration, aeration, reverse osmosis, and the like. Carbon-depleted water is then discharged back into the ocean 105 via a discharge port 103. Often, a CDR facility 101 may be collocated with a power plant, where the power plant use the ingested water 105 for cooling and the CDR facility 101 treats this cooling water before being discharged.

To measure the effects of this human intervention, one or more sampling chambers 107 according to the present invention may be deployed at different depths in the local ocean 105 according to system requirements and constraints. The sampling chambers 107 may be deployed any of a number of well-known mooring mechanisms. In this exemplary environment, each of the chambers 107 is fixed in place via a mooring line 111 that is coupled to a buoy 106 on the surface of the water 107 and an anchor 108 that is affixed to the ocean floor 104 or other fixed anchor point. The mooring lines 111 may also comprise electrical cabling that is employed to couple one or more antennas 109 to each of the chambers 107. In an alternative embodiment, the electrical cabling may also be employed to couple a power source (not shown) to the chambers 107 that includes, but is not limited to, a battery, solar panels, or a cable from a collocated power source. Each of the chambers 107 may transmit collected data and receive commands over one or more wireless links 110 to a CDR monitoring and modeling center (not shown) where the data is employed as inputs to one or more MRV models to measure, report, and validate the effects of CDR at the deployed location.

Operationally, the sampling chambers 107 may be pre-programmed or directed to take samples of the ocean water 105, to allow those samples to equilibrate, to measure one or more fluid properties (e.g., dissolved oxygen, total alkalinity, chlorophyll, temperature, conductivity, turbidity, partial pressure carbon dioxide (pCO2), and potential of hydrogen (pH)). Sensors in the sampling chambers 107, discussed in more detail below, are used to measure those properties where equilibration time of at least one of the sensors is significantly greater than the temporal variability of a corresponding parameter that is measured. The measured properties are then stored locally and may be periodically transmitted to the monitoring and modeling center via the wireless links 110. The wireless links 110 may comprise satellite links, cellular links, or even hard-wired communication links.

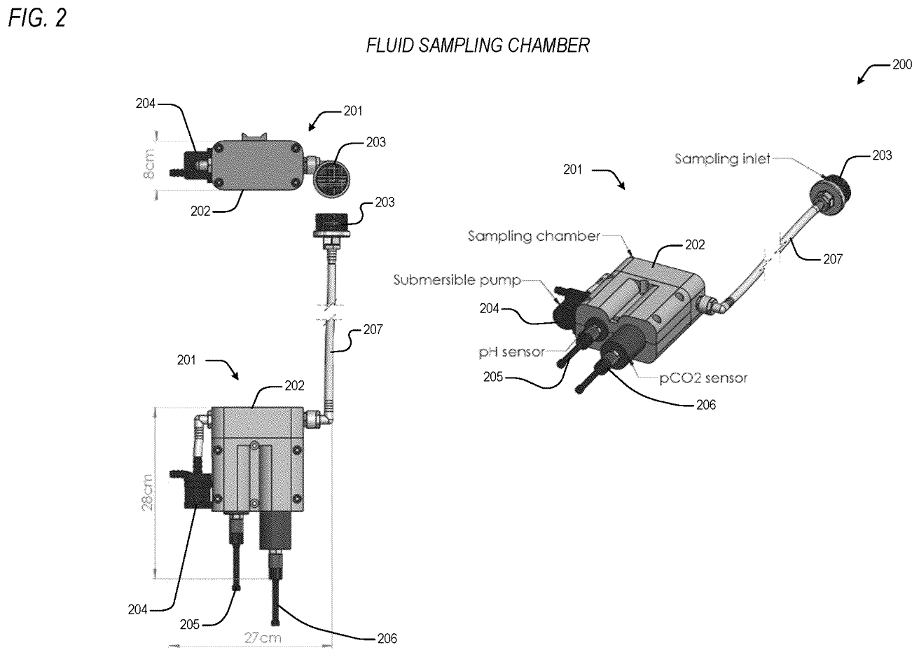

Referring to FIG. 2, a diagram 200 is presented depicting a top view, a side view, and a perspective view of an exemplary fluid sampling chamber 201 according to the present invention. The chamber 201 may comprise a measuring chamber 202 that is coupled to a controlled submersible pump 204, a first sensor (“probe”) 206, a second probe 205, and an optional inlet tube 207. A controllable sampling inlet 203 is coupled to the inlet tube 207.

In one embodiment, the measuring chamber 202 forms a single internal volume and may be manufactured from Delrin or another material having similar properties for underwater use in a saline environment and may be approximately 20 cm×20 cm×8 cm dimensionally, though other sizes are contemplated. Preferably, the internal volume of the chamber 202 for performing fluid properties measurements is roughly 100 milliliters. The measuring chamber 202 is sealed from the environment except for unidirectional flow valves (not shown) located at a chamber inlet (not shown) that is coupled either to the optional inlet tube 207 in an embodiment that employs an inlet tube 207 or the sampling inlet 203 itself in an embodiment that does not employ the inlet tube 207. Provision of the optional inlet tube 207 allows for the sampling inlet 203 to be positioned at various locations within a fluid plume without the need to reposition the chamber 201. The inlet tube 207 may further comprise a copper mesh (not shown), an ultraviolet light (not shown), or other anti-fouling apparatus (not shown) to limit biofouling of its internal surfaces and sensor interfaces. The inlet tube 207 and interior surfaces of the chamber 202 may be formed from or coated with materials that discourage biological growth. In certain embodiments, an automatic cleaning or light-based sterilizing process may occur between sampling cycles when the chamber 202 is sealed. Advantageously, these measures reduce maintenance requirements and extend deployment duration while preserving measurement quality in environments with high biological activity. In one embodiment, the light-based sterilizing process may employ a controllable ultraviolet light to clean the chamber 202.

In another embodiment, the chamber 202 may comprise a flexible bladder (not shown) disposed within the single internal volume chamber 202. The bladder may assist in the flushing process by expanding or contracting to draw in or expel fluid samples without introducing air. In yet another embodiment, the flexible bladder may also form two or more separate internal volumes, allowing the chamber to hold or compare two or more discrete fluid samples. In such configurations, each side of the bladder may be exposed to fluid drawn from one of two or more separate sampling inlets 203 deployed at different locations or depths, enabling paired measurements under matched conditions. In one embodiment, samples from these bladder volume may utilize dedicated sensors 205-206 or the sensors 205-206 may be shared to perform measurement from each of the bladder sides. The timing of flushing and sampling may be pre-programmed or may be controlled as a function of stability of one or more sensor readings. Once the conditions for equilibrium or stability are reached, the chamber may automatically begin or end flushing or measurement cycles.

The chamber 202 may further comprise a mixer (not shown) positioned near the head of the chamber 202 to ensure homogeneity of the sampled fluid. In one embodiment, the mixer may comprise a magnetic or mechanical stirring element that operates to create gentle, stirring of a sample without affecting pressure balance. The mixer may operate periodically or continuously during equilibration to improve measurement accuracy and ensure uniform sensor exposure. Advantageously, the mixing process enables the removal of small bubbles or gradients that can interfere with optical or electrochemical measurements. The pump 204 may regulate the direction and rate of flow for flushing, filling, or sealing cycles. The pumping action may also cooperate with the bladder and the mixer to maintain stable pressure and prevent contamination during transitions.

To clearly teach the present invention, the chamber 201 in the diagram 200 is shown with a pH sensor 205 and a pCO2 probe 206; however, the present inventors note that configuration of the chamber 201 may be easily adapted by those skilled in the art to accommodate a plurality of sensors of different types along with a plurality of pumps 204 and corresponding inlets 203 as are disclosed above. In addition, the present inventors note that the configuration of the chamber 201 may be easily adapted to include one or more sensors that each measure more than a single parameter, such as a multiparameter sonde. In one embodiment, the chamber 201 may comprise a combination of single-parameter probes 205-206 and multiparameter sondes. Though shown on the outside of the chamber 201, the pump 204 may be integrated internally. In embodiments where the chamber 201 is deployed near the surface, the antenna 109 and power source may also be disposed internally.

The chamber 201 may further comprise control electronics (not shown), described in more detail below, that include a power, a processor (“CPU”), a memory, input/output circuits, and a communications circuit. The input/output circuit may be coupled to the pump 204, the unidirectional flow valves, the first sensor 206, and the second sensor 205. The communications circuit may comprise any well-known wired or wireless communication apparatus including, but not limited to, RS-232, Zigbee, IEEE 802.15, Bluetooth, and the like, which are employed to communicate with the CDR monitoring and modeling center. The power supply may comprise a battery or a power conditioner that is coupled to an external source of power (e.g., AC mains, larger battery, solar panel, wave energy converter, tidal energy device, thermal energy converter, etc.). The memory 208 may comprise a combination of both non-transitory and transitory memory. The memory 208 may additionally comprise an operating system (OS), such as Unix, MacOS, Android, iOS, MS Windows, etc. and one or more application programs that implement functions of the chamber 201 by controlling elements therein as further described below. In various embodiments, the one or more application programs are configured to perform the functions that are stored in the non-transitory storage memory, transferred to the transitory storage memory at run time, and then executed by the one or more CPUs.

In operation, the controlled submersible pump 204 is employed to draw a fluid sample from a specified location and time into the measuring chamber 202, where the sample is allowed to equilibrate under optional use of a mixing element, and then is exposed to one or more sensors 205-206 to measure the desired parameters, such as, but not limited to, the first probe 205 and the second probe 206. The one or more application programs may comprise instructions that are configured to execute pre-determined or commanded (via the wireless links 110) sampling schedules and the instructions may direct the processor to control the pump 204 and the acquisition rate of the probes 205-206 by opening and closing the pump 204. In one embodiment, the sampling cycle is as follows: the sampling chamber 202 is first flushed at a high flow rate to dislodge any fouling from the interior, including the optional inlet tube 207. Next, flow rate of the pump 204 is reduced to draw in a sample to be measured in the chamber 202 for a short while until the chamber volume is determined to be filled with the targeted sample based on calculations from the volumetric flux. Unidirectional flow valves seal the chamber 202 when the pump 204 is turned. Next, the sensors 205-206 are activated to start measuring the sample, which allows them to reach equilibrium or conduct one or more discrete measurements of the sample, after which a pre-determined number of data points are collected and optionally averaged over to reduce measurement noise. At the end of the measurement time, the next cycle begins at a scheduled or commanded rate. The measurement data may be transmitted via the wireless link 110 to the CDR monitoring and modeling center. In one embodiment, the data is transmitted when a measurement is determined. Alternatively, measurements may be stored in the internal memory and transmitted to the CDR monitoring and modeling center periodically. Another embodiment may comprise an external data logger or recording device (not shown) in place of the antenna 109, where personnel periodically are deployed to access stored measurements.

The chamber 201 according to the present invention may further be configured to perform a more complete flushing and refilling sequence between measurement cycles. This may include forward and reverse flow steps or short purge intervals to ensure a previous sample is fully removed. The improved flushing control reduces contamination, prevents trapped bubbles, and maintains accuracy.

Advantageously, the present invention provides for collocated and reliable measurements of fluid parameters in highly variable environments such as, but not limited to, the ocean, rivers, outfalls or discharges without the need for water samplers and complicated logistics for sample preservation. This fluid sampling chamber 201 according to the present invention enables the use of existing sensors 205-206 that benefit from an extended exposure to a fluid sample or repeat measurement of the same parameter with the same sensor to increase accuracy and characterize the uncertainty of the measurement. As noted above, the chamber 201 can be further comprise one or more off-the-shelf sensors for measurement of other parameters, and can be seamlessly integrated with different marine platforms, such as, but not limited to moorings, buoys, landers, unscrewed surface vessels (USV's), autonomous underwater vehicles (AUV's) and remotely operated vehicles (ROV's), ships, towed vehicles, etc. One advantage of the present invention is that operation of the chamber 201 does not require specialized personnel, thus making it ideal for long term deployment.

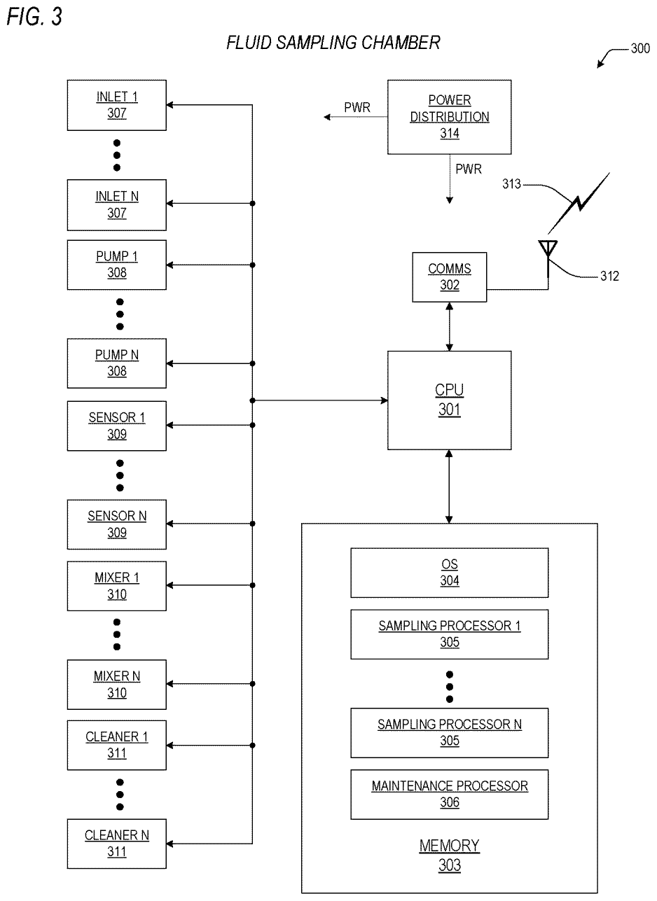

Now turning to FIG. 3, a block diagram is presented of an exemplary fluid sampling chamber 300 according to the present invention, such as the fluid sampling chamber 201 of FIG. 2. The chamber 300 may comprise central processing unit (CPU) 301 that is coupled to a memory 303 having both transitory and non-transitory memory components therein. The CPU 301 is also coupled to a communications circuit 302 that couples the chamber 300 to an antenna 312, which enables the chamber 300 to communicate with a CDR center (or data logger) via one or more wireless links 313 as discussed above. Alternatively, the communications circuit 302 may directly link the chamber 300 to the CDR center (or data logger) via a hard-wired link without a requirement for an antenna 312.

Coupled to the CPU 301 are one or more inlet controls INLET 1 307-INLET N 307, one or more pump controls PUMP 1 308-PUMP N 308, one or more sensor controls SENSOR 1 309-SENSOR N 309, one or more mixer controls MIXER 1 310-MIXER N 310, and one or more cleaner controls CLEANER 1 311-CLEANER N 311. The number of controls 307-311 is determined by configuration requirements. In one embodiment, the CPU 301 is coupled to one inlet control 307, one pump control 308, 2 sensor controls 309, one mixer control 310, and one cleaner control 311, where the sensor controls 309 are employed to interface and communicate with a pH sensor and a pCO2 sensor.

The chamber 300 may also comprise power distribution circuits 314 that provide for conditioning and distribution of the voltages and currents required to power all of the chamber components 301-312. In one embodiment, the power distribution circuits 314 may comprise a non-rechargeable battery. In another embodiment, the power distribution circuits may comprise a rechargeable battery where the battery may be recharged via a port accessible on a corresponding mooring point, such as a buoy that has a solar panel affixed thereto.

The memory 303 may include an operating system 304 such as, but not limited to, Microsoft Windows, Mac OS, Unix, and Linux, where the operating system 304 is configured to manage execution by the CPU 301 of program instructions that are components of one or more application programs. In one embodiment, a single application program comprises a plurality of code segments 305-306 resident in the memory 303 and identified as one or more sampling processors SAMPLING PROCESSOR 1 305-SAMPLING PROCESSOR N 305 along with a maintenance processor MAINTENANCE PROCESSOR 306. Other code segments (not shown) may be provided to perform other functions by the chamber 300 which are not discussed herein in order to clearly teach relevant aspects of the present invention.

In one embodiment, the number of sampling processors 305 is a function of the number of sample chambers 202 disposed therein. As noted above, the chamber 202 may comprise a flexible bladder (not shown) disposed within the chamber 202 that may assist in the flushing process by expanding or contracting to draw in or expel fluid samples without introducing air. In one embodiment, the flexible bladder may also form two separate internal volumes, allowing the chamber to hold or compare two discrete fluid samples, and thus requiring two sample processors 305.

The CPU 301 may execute one or more of the modules 305-306 as required to direct the chamber 300 obtain fluid samples, to enable those samples and corresponding sensors 309 to equilibrate, to take sample readings from the sensors 309, to receive commands via the communications circuit 302, to store and transmit those readings via the communications circuit 302, to flush the chamber 300 (or bladder reservoirs), to (optionally) perform mixing operations of an ingested sample, and to perform cleaning operations and anti-fouling operations.

In operation, the power distribution circuits 314 may power up the chamber components 301-311 to begin execution of the one or more sampling processors 305 and the maintenance processor 306 are coordinated to perform sampling by and maintenance of the chamber 300 in accordance with configurable sampling and maintenance schedules. The configurable sampling and maintenance schedules may be pre-programmed into the memory 303 or may be directed by commands received over the communications circuit 302. Each of the sampling processors 305 direct the CPU to control actuation, flow direction, and flow rate of the pumps 308 and to coordinate opening/closing of the inlets 307 to flush the chamber 300 and to ingest a sample while facilitating homogeneity of sampled fluid via the mixers 310. Following ingestion of a sample, a corresponding sampling processor 305 may direct the CPU 301 to delay reading one or more corresponding sensors 309 to allow for equilibration of the corresponding sensors 309 and then may direct the CPU 301 to read one or more of the corresponding sensors 309 following equilibration, where equilibration times for individual sensors 309 are based upon sensor type and fluid type. Following read of the corresponding sensors 309, sensor readings may be transmitted via the communications circuit 302 or may be stored in the memory 303 and later uploaded via the communications circuit 302. The sampling processors 305 may further direct the cleaners 311 to activate periodically or in coordination with sampling cycles in order to preclude bio-fouling. The maintenance processor 306 may control the inlets 307, pumps 308, mixers 310, and cleaners 311 to perform anti-fouling operations when during periods when samples are not being taken.

The fluid sampling chamber 300 according to the present invention is configured to perform the functions and operations as discussed above and may comprise both transitory and non-transitory stores 303. The one or more application programs may be transferred from the non-transitory stores 303 and cached within the transitory storage 303 for speed of execution at run time. The chamber may comprise digital and/or analog logic, circuits, devices, or microcode (i.e., micro instructions or native instructions), or a combination of logic, circuits, devices, or microcode, or equivalent elements that are employed to execute the functions and operations according to the present invention as noted herein. The elements employed to accomplish these operations and functions within the chamber 300 may be shared with other circuits, microcode, etc., that are employed to perform other functions and/or operations within the chamber 300. According to the scope of the present application, microcode is a term employed to refer to a plurality of micro instructions. A micro instruction (also referred to as a native instruction) is an instruction at the level that a unit executes.

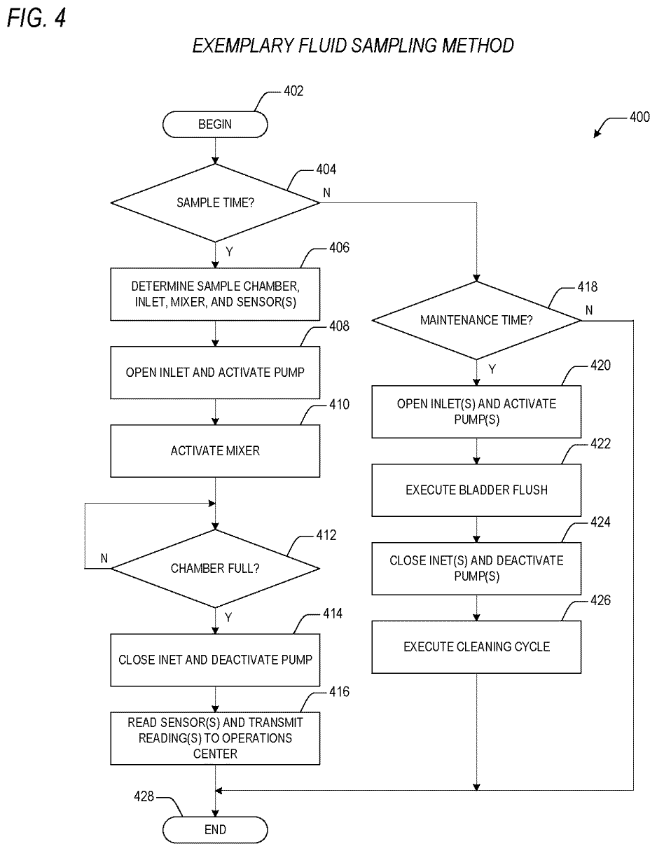

Now referring to FIG. 4, a flow diagram 400 is presented showing an exemplary method for fluid sampling according to the present invention. Flow begins at block 402 where a fluid sampling chamber according to the present invention is powered up and operational in accordance with desired configuration. Flow then proceeds to block 404.

At block 404, an evaluation is made to determine if it is time to perform a sampling operation. If so, then flow proceeds to block 406. If not, then flow proceeds to decision block 418.

At block 406, the sampling processor 305 determines which sample chamber (in a multi-chamber bladder, inlet, mixer, and sensor(s) to control for ingesting and measuring properties of the sample. Flow then proceeds to block 408.

At block 408, the sampling processor may control an inlet and pump corresponding to the selections determined at block 406 and may flush the selected chamber and then begin ingesting a sample from the inlet. Flow then proceeds to block 410

At block 410, the sampling processor 305 may optionally activate a mixer corresponding to the selections determined at block 406 to ensure homogeneity of the sample. Flow then proceeds to decision block 412.

At decision block 412, an evaluation is made to determine if the chamber is full. If not, then flow proceeds to decision block 412, thus waiting for the chamber to fill. If so, then flow proceeds to block 414.

At block 414, the sampling processor 305 closes the inlet and deactivates the pump for a period of time to allow corresponding sensor(s) to equilibrate. Flow then proceeds to block 416. At block 416, the sampling processor reads the sensor(s) and may transmit the reading(s) to the operations center. Alternatively, sensor readings are stored in memory and may be additionally pre-processed (e.g., averaging, discard of out-of-range readings, interpolation of missing readings, etc.) prior to transmission to the operations center. Flow then proceeds to block 428.

At decision block 418, an evaluation is made to determine if it is time to perform a periodic maintenance operation. If not, then flow proceeds to block 428. If so, then flow proceeds to block 420.

At block 420, the maintenance processor 306 may open one or more inlets and activate one or more corresponding pumps to purge excess fluid and debris from corresponding one or more chambers. Flow then proceeds to block 422.

At block 422, one or more chambers within the internal bladder are flushed, where the flushing operation may change flow directions and flow rate in order to dislodge debris. Upon completion of the flushing operation, flow proceeds to block 424.

At block 424, the maintenance processor 306 closes the one or more inlets and deactivates the corresponding pumps. Flow then proceeds to block 426.

At block 426, the maintenance processor 306 may execute a cleaning cycle. In the case where the cleaner is an ultraviolet lamp, the maintenance processor 306 may turn the lamp on for a prescribed time period and then turn it off. In the case that the chamber is configured with a cleaner that does not require control (e.g., anti-fouling interior coatings, copper mesh, etc.), then this step is skipped. Flow then proceeds to block 428.

At block 428, the method completes.

The present inventors note that the method described above with reference to FIG. 4 only performs one cycle of sampling or maintenance. For continuous operation, rather than ending the flow at block 428, flow rather proceeds to block 404 where a next cycle of sampling or maintenance is performed.

The present inventors also note that the method discussed above presumes use of a plurality of chambers disposed in a chamber bladder along with a corresponding plurality of inlets, mixers, and sensors. Such an example illustrates operation of the present invention at full configuration; however, it is further noted that a bladder is not required (thus requiring a single inlet and pump), nor is a mixer required, nor is a cleaner required. Environmental and operational constraints are deemed to dictate individual configurations of the fluid sampling chamber according to the present invention.

Portions of the present invention and corresponding detailed description are presented in terms of software, or algorithms and symbolic representations of operations on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer program product, a computer system, a microprocessor, a central processing unit, or similar electronic computing device, that manipulates and transforms data represented as physical, electronic quantities within the computer system's registers and memories into other data similarly represented as physical quantities within the computer system memories or registers or other such information storage, transmission or display devices. The devices may comprise one or more CPUs that are coupled to a computer-readable storage medium. Computer program instructions for these devices may be embodied in the computer-readable storage medium. When the instructions are executed by the one or more CPUs, they cause the devices to perform the above-noted functions, in addition to other functions.

Note also that the software implemented aspects of the invention are typically encoded on some form of program storage medium or implemented over some type of transmission medium. The program storage medium may be electronic (e.g., read only memory, flash read only memory, electrically programmable read only memory), random access memory magnetic (e.g., a floppy disk or a hard drive) or optical (e.g., a compact disk read only memory, or “CD ROM”), and may be read only or random access. Similarly, the transmission medium may be metal traces, twisted wire pairs, coaxial cable, optical fiber, or some other suitable transmission medium known to the art. The invention is not limited by these aspects of any given implementation.

The particular disclosed above are illustrative only, and those skilled in the art will appreciate that they can readily use the disclosed conception and specific embodiments as a basis for designing or modifying other structures for carrying out the same purposes of the present invention, and that various changes, substitutions and alterations can be made herein without departing from the scope of the invention as set forth by the appended claims. For example, components/elements of the systems and/or apparatuses may be integrated or separated. In addition, the operation of the systems and apparatuses disclosed herein may be performed by more, fewer, or other components and the methods described may include more, fewer, or other steps. Additionally, unless otherwise specified steps may be performed in any suitable order.

Although specific advantages have been enumerated above, various embodiments may include some, none, or all of the enumerated advantages.

1. A fluid sampling apparatus, comprising:
a sampling chamber, configured to receive a fluid sample;
at least one controllable inlet fluidically coupled to the sampling chamber;
a pump, configured to draw fluid through the inlet and into the sampling chamber;
at least one sensor, disposed to measure at least one property of the fluid sample within the sampling chamber; and
a processor, configured to control operation of the inlet, the pump, and the at least one sensor to obtain a measurement of the fluid sample following equilibration of the fluid sample.

2. The apparatus as recited in claim 1, further comprising a flexible bladder defining the sampling chamber.

3. The apparatus as recited in claim 1, further comprising:
a mixer configured to homogenize the fluid sample within the sampling chamber.

4. The apparatus as recited in claim 1, further comprising:
a cleaner configured to reduce or remove biological fouling from an interior surface of the sampling chamber.

5. The apparatus as recited in claim 4, wherein the cleaner comprises a controllable ultraviolet light source, configured to illuminate interior surfaces of the flexible bladder between sampling cycles.

6. The apparatus as recited in claim 1, wherein the at least one sensor comprises at least one of a pH sensor, a pCO2 sensor, a dissolved oxygen sensor, a conductivity sensor, or a multiparameter sonde.

7. The apparatus as recited in claim 1, wherein the processor is configured to execute a flushing cycle comprising forward and reverse pump operation prior to drawing the fluid sample.

8. A fluid sampling chamber for in-situ measurement of heterogeneous aquatic fluids, comprising:
a sealed measuring chamber comprising a flexible bladder, configured to define an internal sampling volume;
at least one controllable sampling inlet coupled to the flexible bladder;
a pump, configured to flush the flexible bladder and draw a fluid sample into the internal sampling volume;
a mixer, positioned to homogenize the fluid sample within the internal sampling volume;
at least one sensor having an equilibration time greater than temporal variability of at least one fluid property external to the chamber;
a cleaner;
at least one unidirectional flow valve configured to seal the internal sampling volume during equilibration; and
a processor configured to:
perform a flushing cycle prior to sampling;
fill the internal sampling volume with the fluid sample;
activate the mixer to homogenize the fluid sample;
allow the at least one sensor to equilibrate while the internal sampling volume is sealed;
obtain at least one measurement from the at least one sensor; and
activate the cleaner between sampling operations.

9. The fluid sampling chamber as recited in claim 8, wherein the flexible bladder comprises at least two internal sampling volumes configured to hold corresponding discrete fluid samples.

10. The fluid sampling chamber as recited in claim 8, wherein the mixer comprises a magnetic or mechanical stirring element.

11. The fluid sampling chamber as recited in claim 8, wherein the processor is configured to determine a stability condition for the at least one sensor and to end an equilibration period upon meeting the stability condition.

12. The fluid sampling chamber as recited in claim 8, further comprising:
an inlet tube disposed upstream of the controllable sampling inlet, the inlet tube comprising at least one anti-fouling feature selected from a copper mesh, ultraviolet emitter, or biofouling-resistant coating.

13. The fluid sampling chamber as recited in claim 8, wherein the pump is configured to operate at a first flow rate for flushing and a lower second flow rate for sample ingestion.

14. The fluid sampling chamber as recited in claim 8, wherein the flexible bladder is configured to expand or contract to draw in or expel fluid without introducing air into the sampling chamber.

15. A method for sampling a fluid, comprising:
drawing a fluid sample into a sampling chamber through at least one controllable inlet;
allowing at least one sensor disposed in the sampling chamber to measure at least one property of the fluid sample; and
controlling operation of the inlet, a pump, and the at least one sensor using a processor to obtain the measurement.

16. The method as recited in claim 15, further comprising flushing the sampling chamber prior to drawing the fluid sample.

17. The method as recited in claim 15, further comprising homogenizing the fluid sample within the sampling chamber using a mixer.

18. The method as recited in claim 15, further comprising activating a cleaner to reduce biological fouling inside the sampling chamber between sampling operations.

19. The method as recited in claim 15, further comprising deploying the sampling chamber at a depth in an aquatic environment using a mooring line coupled to a buoy and an anchor.

20. The method as recited in claim 15, wherein the at least one sensor comprises at least one of a pH sensor, a pCO2 sensor, a dissolved oxygen sensor, a conductivity sensor, or a multiparameter sonde.
